(19) United States
(12) Patent Application Publication
(10) Pub. No.: US 2022/0133772 A1
(43) Pub. Date: May 5, 2022

(54) MEDICATION

(71) Applicant: PlantTec Medical GmbH, Lüneburg (DE)

(72) Inventors: Joachim LAAS, Bad Bevensen (DE); Kai-Oliver MUTZ, Hannover (DE)

(21) Appl. No.: 17/525,465

(22) Filed: Nov. 12, 2021

Related U.S. Application Data
(63) Continuation of application No. 16/320,047, filed on Aug. 4, 2020, now abandoned.

(30) Foreign Application Priority Data
Jan. 4, 2018 (DE) 10 2018 000 009.6

(51) Int. Cl. A61K 31/718; A61P 17/02; A61K 31/194

(52) U.S. Cl. CPC A61K 31/718; A61K 9/06; A61K 31/194; A61P 17/02

(57) ABSTRACT

A medication is disclosed, and the medication includes carboxymethylated starch. The medication includes a citrate and/or citric acid for reducing postoperative adhesions. The citrate and/or citric acid is/are situated on the surface of granules of the starch, and includes 1 to 20 percent by weight of citrate and/or citric acid relative to the starch. A method of treating postoperative adhesions by administering the topical medication for reducing postoperative adhesions to a postoperative adhesion of a subject, and a use of the topical medication.

The invention relates to a medicinal material. The invention also relates to a method for producing a medicinal material, a use of the demonstrated medicinal material, an additional use as well as an inventive method.

US 2002/0169144A1 discloses a pharmaceutical composition that comprises a cross-linked polyanionic polysaccharide, such as carboxymethyl amylose and a citrate buffer, the composition preventing the formation of surgical adhesions.

US 2004/0153040 A1 discloses a wound dressing comprising a layer of hydrophilic polymer foam and a hydrogel layer used to contact the surface and as an anti-adhesion ingredient, wherein the hydrogel layer may contain carboxymethyl starch, wherein the polymer foam may be cross-linked with citric acid and the wound bandage is used during a postoperative treatment.

WO 2016/098057 A1 discloses a chitosan hydrogel, which includes citric acid and carboxymethyl starch, the hydrogel being used to counteract postoperative adhesions.

DE 10 2013 211 316 A1 discloses a hemostatic agent comprising carboxymethyl starch, which is chemically cross-linked by citric acid.

WO 2016/100861 A1 discloses a hemostatic composition comprising cross-linked modified amylopectin, icodextrin or maltodextrin, which includes carboxymethyl starch, carboxymethyl maltodextrin or carboxymethyl icodextrin, as well as sodium citrate as a constituent of an aqueous diluent.

Organs and tissues in the body that are covered with a mesothelium (heart, lungs and intra-abdominal organs, parietal wall of the pericardium, thorax and stomach cavity) are normally able to freely move and are separate from each other, even when their surfaces are in close contact. Articular cavities are covered with the synovium, which is essential there for the free movement of bones and joints. This also explains synovial structures in the case of tendons and nerves. One reaction to surgical interventions in such cavities and synovial structures is the development of adhesions as part of the healing process, which is necessary in order to close a wound and, for example, to cover it against infections. Adhesions may often cause problems, however. Adhesions may appear as slight accumulations that are easy to dissolve or as bands of fibrous, sometimes vascularized tissue (consisting of blood vessels and connective tissue). They may even appear as complete adhesions, which abnormally fix the organs permanently. Adhesions may cause chronic pain, disorders of the stomach—intestine passage or of the ileus and thus lead to further surgical interventions or even death. Adhesions increase the risk during operations due to bleeding during removal of the adhesions. Moreover, the areas removed of adhesions are hearths for the development of new adhesions. Medical complications aside, the treatment of adhesions is a significant socioeconomic problem. For this reason, a safe and low-risk prevention of adhesions is one of the main goals of modern surgery. Adhesions in synovial structures may cause chronic pain and movement impairment. Aside from the medical aspect, there are considerable socioeconomic aspects here as well.

Operations are the most frequent cause for the formation of adhesions and they may be a serious complication. The predisposing factors include mechanical injuries of mesothelial (mesothelium-like such as, for example, synovial) surfaces or peritoneal ischemia due to manipulation and retraction of tissues during operation.

The incidence of postoperative adhesion formation is estimated today at 67% to 93%. At present, there is a variety of barriers on the market for reducing this problem. While the principle is invariably the physical separation of wound areas, the products differ considerably due to their make, structure and composition. There are barriers or rinse solutions produced from cellulose or polytetrafluorethylene, icodextrin, hyaluronic acid as well as carboxymethyl cellulose.

In addition to commercial products for reducing postoperative adhesions, other medicinal materials are also conceivable. The most promising solutions appear to be the use of modified starches as mechanical barriers, as well as the use of citrate for inhibiting the formation of fibrin. Hoffmann et al. (2009), for example, report that products for hemostasis may also reduce postoperative adhesions. Starch-based products for hemostasis, in particular, significantly reduce the degree of adhesions as compared to other “conventional” topical products for hemostasis. With Arista™ AH (disclosed by the patent family U.S. Pat. No. 6,060,461), consisting of microporous starch granules, it is possible to achieve a maximum 50% reduction of postoperative adhesions in the rat model. The patent family WO2009/091549 discloses, in particular, a hemostatic material, which in this case is a modified starch having a water absorption capacity of no lower than the simple inherent weight, having a molecular weight of greater than 15,000 Daltons and having a starch granule diameter of 10 to 1,000 μm, at least 95% of the starch granules having a diameter between 30 μm and 500 μm. This modified starch is biocompatible, absorbable and is degraded through the action of amylases and carbohydrases. Methods with regard to application are disclosed, in which the hemostatic material is applied to tissue, for example, in the form of powder or as a gel and is used, inter alia, for the purpose of hemostasis and adhesion prophylaxis. In two publications, Pope et al. (1914 and 1916) report on a reduction of postoperative adhesions due to the use of citrate. It was shown in 60 experiments on rabbits that 2% to 4% citrate in water or saline solution resulted in fewer adhesions. This was confirmed in clinical application in 400 abdominal operations and it was shown that a clear improvement with respect to the development of adhesions was evident as result of citrate.

Alternative solutions to the problem of postoperative adhesions are always desirable. The fact, however, that adhesions may be reduced to a never before seen positive degree by modified starch (synergetically improved compared to modified starch as a mechanical barrier or citrate for inhibiting fibrin formation, in each case individually), which contains citrate and citric acid (either from the manufacturing process or through admixture), applied to traumatized mesothelial (or mesothelium-like) surfaces (zones at risk for developing adhesions) and transformed from starch powder to a starch gel, is unique and not yet known.

According to the invention, it was possible to show that this inventive medication, referred to below as medicinal material, is excellently suited for reducing postoperative adhesions in a never before seen way and, according to the invention, synergistically combines for this purpose the mechanical barrier effect of a modified starch gel and the fibrin formation-inhibiting citrate effect.

The following explanations are not limiting in terms of the invention:

The inventive medicinal material consists of carboxymethylated and cross-linked starch, a 1% to 20% portion of which is citrate, since this combination offers never before seen positive results for reducing postoperative adhesions. This applies, in particular, to treatments for reducing postoperative adhesions, in particular, traumatized mesothelial (or mesothelium-like) surfaces (zones at risk for developing adhesions) such as, for example, the pleura in the chest cavity, peritoneum in the belly and pelvic cavity or pericardium serosum in the pericardial cavity, as well as synovial structures of bones, joints, tendons or nerves.

This never before seen positive property for reducing postoperative adhesions is assumed to be based on the following mechanisms:

An efficient tissue separation during wound healing is guaranteed by the inventive medicinal material, applied in gel form or transformed from powder into gel form, on traumatized mesothelial (or mesothelium-like) surfaces (zones at risk for developing adhesions). The fact that the inventive medicinal material is applied in gel form or powder form and, for example, as a film, aids in an effective and reliable barrier effect down to the smallest corner of the traumatized area. Since carboxymethylated and cross-linked starch and citrate are absorbed within a few days, there is no need for a second operation to remove the mechanical barrier. This makes it possible to prevent adhesions because the traumatized areas are covered with the gel barrier and thus physically separated from adjacent or other surfaces. The effect is synergetically reinforced by the fibrin formation-inhibiting citrate effect. Citrate forms insoluble chelate complexes with calcium, as a result of which a formation and stabilization of fibrin polymerization sites in all subsequent stages of fibrin polymerization is achieved. Citrate limits the deposition of fibrin through the bonding of calcium ions, in order in this way to render the subsequent formation of adhesions impossible. As a result, the inventive medicinal material offers two different antiadhesive effects, physical barrier and fibrin polymerization inhibition.

Methods

Forty Lewis rats were used for the study. They were housed under standard conditions, had continuous access to fresh water and were fed with standard nutrition. Their well-being was monitored through daily examinations of observed changes during application. In addition, they were weighed on a daily basis so that deviations from normal body weight development could be determined. All protocols with respect to quality of life of the animals were carried out in compliance with national and European laws.

Ketamine and xylazine were used for the anesthesia. The required level of narcosis was achieved once the flexor-reflexes were suppressed. After shaving, the abdomen was cleaned with ethanol and iodine solutions. Access to the abdomen was achieved by a median laparotomy of approximately 3.5 cm. The induction of adhesions was carried out in accordance with an adhesion model. The cecum was exposed and kept moist. The visceral peritoneum of the cecum was then repeatedly rubbed with a watery gauze over an area 1×2 cm in size. A surface of the parietal peritoneum on the right abdominal wall 1×2 cm in size, including the inner muscle layer was sharply resected. Both resulting defects were drawn in spatial proximity to one another using a 4/0 prolene stitch in order to induce maximum adhesions. The abdomen was closed with a dual layer closing technique using a consecutive stitch. The animals were monitored postoperatively until they were fully awakened.

On the seventh postoperative day, the animals were killed during application. In order not to destroy a potentially formed adhesion, the peritoneal cavity was opened through an incision to the left of the original laparotomy scar. Cecum and abdominal wall were removed for histopathological study.

Ten animals were each randomly assigned to one of the four groups:

1. Control

2. Inventive medicinal material

3. Modified starch without citrate

4. Citrate only

Control animals received only sterile 0.9% saline solution. The medicinal material as well as the modified starch without citrate were administered as 300 mg of powder and transformed on site into a gel with 1.2 mL of sterile 0.9% saline solution.

During the autopsy, the adhesion formation between the defective abdominal wall and the cecum was macroscopically evaluated. For this purpose, the following system was utilized:

| Result | Description |
|---|---|
| 0 | No adhesions |
| 1 | Thin-film adhesions |
| 2 | More than a thin adhesion |
| 3 | Thick adhesion with focus |
| 4 | Thick adhesion with planar attachment |
| 5 | Very thick vascularized adhesions or more than one planar |
| | adhesion |

Adhesion values are represented as arithmetic averages with standard deviations. Since most of the data sets did not follow a Gaussian distribution (determined with the D'Agostino-Pearson normality test), the multiple comparison of the adhesion assessment was carried out using the Kruskal-Wallis-Test, followed by Dunns multiple comparison test for non-parametric data (which use the correction for multiple comparisons with the aid of statistical hypothesis testing). Groups were variously defined as significant if p<0.05.

Results

The tested animals all showed a comparable viability and body weight development. None of the animals had to be prematurely killed due to complications; all 40 animals completed the experiment.

Within the control group, 9 of 10 animals showed massive adhesions, each of which received the maximum number of points, whereas the tenth animal developed no adhesions at all.

None of the animals treated with the inventive medicinal material developed any adhesions, so that all animals were classified in categories 0.

Of the animals treated with the modified starch without citrate, only two developed mild adhesions, which were assessed with 1. The other eight animals treated with the modified starch without citrate developed severe adhesions and were given a 4 or 5.

The animals treated with citrate alone had an average assessment of 2.4.

The following reductions, converted to a percent value, indicate:

1. Control=0%

2. Inventive medicinal material=100%

3. Modified starch without citrate=28%

4. Citrate only=52%

An improved reduction of adhesions is achieved with an inventive medicinal material of this type made of carboxymethylated and cross-linked starch and citrate as a result of the citrate adsorbing on the surface of the starch granules. Only a completely unexpected and unforeseeable synergistic effect can explain why this causes a reduction of adhesions not only of 50% but of even 100%. The transformation of carboxymethylated and cross-linked starch into a gel through liquid absorption slows the release of citrate. Citrate is initially dissolved from the starch granule surface, thereafter dissolved in an organic gel medium and the citrate ions are then conducted across the gel-to-liquid interface. The high water solubility of citrate at the interface produces a concentration gradient along the gel, which forces the ions to move. For this reason, this starch-adsorbed citrate is effective over a significantly longer period of time than pure citrate as salt or in solution. Ultimately, this synergy between carboxymethylated and cross-linked starch and citrate prolongs the fibrin polymerization inhibition effect and thereby reinforces the reduction of adhesions as compared to the sum of the individual operative effects.

In this connection, the inventive medicinal material is also suitable for hemostasis and for the treatment of wounds.

The medicinal material is advantageously present in gel form or is transformed from powder into gel form, the medicinal material according to the present invention advantageously containing water and/or at least one salt, for example, and, in particular, sodium chloride. In practice, it has been positively shown that the starch has a molecular weight of 500,000 Daltons to 11,000,000 Daltons and/or the starch has a starch granule diameter of 30 μm to 100 μm.

In the simplest form of the inventive method for producing said inventive medicinal material, the aforementioned modified starch is provided in gel form or else is prepared with water and, optionally salts, to form a gel.

According to the invention, the use of the inventive medicinal material for reducing postoperative adhesions as well as for hemostasis and for treating wounds is also claimed.

The use of carboxymethylated and cross-linked starch for producing a medicinal material for reducing postoperative adhesions, in particular, if it involves the applications as mentioned above, is also claimed.

Finally, a method for reducing postoperative adhesions is also claimed, in which the inventive medicinal material is used, in particular, when said medicinal material is topically applied. In other words, when postoperative adhesions are treated by applying the inventive topical medication to a subject in need thereof.

The administration of the inventive medicinal material, respectively, the modified and cross-linked starch, either as a gel or transformed to gel with water and, optionally, salts, means that a gel then guides the development of postoperative adhesions on traumatized mesothelial (or mesothelium-like) surfaces (zones at risk for developing adhesions) according to the mechanism described.

In histological studies, there are hardly any to no postoperative adhesions apparent in previously traumatized mesothelial (or mesothelium-like) surfaces after one week. Moreover, a complete wound closure and a very good submesothelial healing is apparent. Both are brought about by the application of the inventive medicinal material in gel form, i.e., a form of the inventive medicinal material that shows residues of starch granules, as well as macrophages and giant cells, fibroblasts and new connective tissue.

The never before seen positive reduction of postoperative adhesions is caused by the presence of the inventive medicinal material, which acts as a mechanical barrier and also makes fibrin polymerization inhibition possible.

The following embodiment explains in a non-limiting manner and by way of example the inventive medicinal material.

The following characterizations of the carboxymethylated and cross-linked starch are advantageous embodiments.

The inventive medicinal material is carboxymethylated and cross-linked starch, which contains relatively constant proportions of approximately 20% by weight of amylose and 80% by weight of amylopectin. The amylose is more amorphous, the amylopectin constitutes the crystalline portion of the starch. The carboxymethylated and cross-linked starch is present in the form of a salt-starch glycolate. It is generally a sodium salt of a carboxymethyl ether of starch. The carboxymethylated and cross-linked starch may contain up to 10% by weight of salt. It is generally a sodium salt. The pH value falls between 3 RE and 7.5 RE (RE=relative units). The percentage portion of salt, which is bonded to the modified starch, falls between 2% by weight and 5% by weight. The modified starch is present as white or nearly white, fine, flowable powder. The powder is very hygroscopic and is practically insoluble in methylene chloride, for example. The result is a translucent suspension in water. The powder consists of irregularly formed oval and pear-shaped particles, which are 30 μm to 100 μm in size. The surface is typically approximately 0.2 m²/g. Occasionally occurring concatenations of particles consist of two to four particles. The particles have an excentric hilus and clearly visible concentric grooves. Between intersecting Nicol prisms, the particles show a clear black cross at the hilus. No crystals are visible on the surface of the particles. The particles exhibit a significant swelling when in contact with water or aqueous saline solutions. This swelling may amount to approximately as much as 30 times the inherent weight. The molecular weight is typically around 500,000 to 11,000.000 Daltons. A starch particle consists of 4·10¹⁰ to 2·10¹² amylose molecules and approximately 5·10′ to 1·10¹⁰ amylopectin molecules. The scale of the cross-linking is approximately around 25% to 45%.

The following production description of the carboxymethylated and cross-linked starch are advantageous embodiments.

The carboxymethylated and cross-linked starch is produced generally in two steps, in each case in a suspension, initially the cross-linking, then the carboxymethylation. The substitution is inhomogeneous since the reactions take place in the solid phase. Outer regions of the starch are more heavily affected by the modifications than the core regions of starch granules. The reactions are influenced by diffusion within the starch structure and by accessibility to the starch structure. Amorphous regions are more easily accessible than crystalline regions, which are less heavily modified. Both reactions are unspecific from a chemical perspective and (to the extent diffusion resistance or steric resistance are present) have no preferred reaction pattern for the individual hydroxyl groups of a glucose unit. In theory, the degree of substitution is at 3 (all three hydroxyl groups of a glucose unit would be substituted). In reality, however, the degree of substitution is probably about 0.2 to 0.4, i.e. approximately 3 for each. Glucose unit is a hydroxy group substituted (not also considered are the cross-linkings). This degree of substitution is a result of the values for the bonded salt. At the end of the reactions, the citrate in the form of citric acid is added to neutralize the reaction solution and is retained as residue in the carboxymethylated and cross-linked starch.

Possible starch gels use water, isotonic sodium chloride solution, hypotonic, isotonic or hypertonic saline solution as the liquid phase with the following cations: sodium, potassium, ammonia, magnesium, calcium, iron (II), iron (III), aluminum; and the following anions: fluoride, chloride, bromide, iodide, oxide, sulfide, carbonate, sulfate, phosphate, nitrate, chromate, permanganate, ferricyanide (II). Ringer's solution, Ringer acetate solution and Ringer lactate solution may also be used as a liquid phase.

1. A method of treating postoperative adhesions by administering a topical medication for reducing postoperative adhesions to a postoperative adhesion of a subject in need thereof, the topical medication for reducing postoperative adhesions including:
a) carboxymethylated starch;
b) a citrate and/or citric acid;
c) the citrate and/or citric acid is/are situated on the surface of granules of the starch; and
d) including 1 to 20 percent by weight of citrate and/or of citric acid relative to the starch.

2. A use of:
a) carboxymethylated starch; and
b) citrate/or citric acid for producing a topical medication for reducing postoperative adhesions by administering the topical medication to a postoperative adhesion of a subject in need thereof.

3. Use according to claim 2, wherein:
a) the treatments are for reducing postoperative adhesions.

4. Use according to claim 2, wherein:
a) the carboxymethylated starch is cross-linked.

5. Use according to claim 2, wherein:
a) the topical medication is used for reducing postoperative adhesions on mesothelial or mesothelium-like surfaces.

6. Use according to claim 2, wherein:
a) the topical medication is used to treat mesenchymal organs or for wound healing of mesenchymal organs, tissues and organs in serous cavities or in epithelial tissue.

7. Use according to claim 2, wherein:
a) the mesenchymal organs are loose, tight, and reticular connective tissue, bones and cartilage, smooth musculature and cardiac muscle, kidneys and adrenal cortex, hematopoietic system, blood, and lymph glands.

8. Use according to claim 2, wherein:
a) the topical medication is used for treating epithelial defects.

9. Use according to claim 2, wherein:
a) the topical medication is used for treating wounds.

10. Use according to claim 2, wherein:
a) the topical medication is used for hemostasis.

11. Use according to claim 2, wherein:
a) the topical medication is used for reducing postoperative adhesions.

12. Use according to claim 2, wherein:
a) the topical medication is active in gel form.

13. Use according to claim 2, wherein:
a) the topical medication contains water and/or at least one salt.

14. Use according to claim 2, wherein:
a) the starch has a molecular weight of 500,000 to 11,000,000 Daltons and/or the starch has a size of 30 μm to 100 μm.

15. Use according to claim 2, wherein:
a) the carboxymethylated and cross-linked starch is one that contains approximately 20 percent by weight of amylose and approximately 80 percent by weight of amylopectin.

16. Use according to claim 2, wherein:
a) the starch is present as a salt-starch glycolate.

17. Use according to claim 2, wherein:
a) the starch contains up to 10 percent by weight of salt.

18. Use according to claim 2, wherein:
a) the pH value is between 3 RU and 7.5 RU.

19. Use according to claim 2, wherein:
a) the proportion of salt bonded to the starch is between 2 and 5 percent by weight.

20. Use according to claim 2, wherein:
a) the scale of the cross-linking is between 25% and 45%.
